(19) United States
(12) Patent Application Publication
(10) Pub. No.: US 2007/0125390 A1
(43) Pub. Date: Jun. 7, 2007

(54) Method of evaluating the effects of exogenous and endogenous factors on the skin

(76) Inventors: Isabelle Afriat, New York, NY (US); Miriam Rafailovich, Plainview, NY (US); E. Guan, Stony Brook, NY (US)

Correspondence Address: THE ESTEE LAUDER COS, INC, 155 PINELAWN ROAD, STE 345 S, MELVILLE, NY, 11747, US

(21) Appl. No.: 11/296,236

(22) Filed: Dec. 7, 2005

(51) Int. Cl. A61B 19/00; G06K 9/00

(52) U.S. Cl. 128/898; 382/128

(57) ABSTRACT

The present invention provides non-invasive, in vivo, DISC-type methods of evaluating structural changes in skin due to a variety of exogenous or endogenous factors. It is possible to develop quantitative and qualitative characterizations of skin. For example, the skin may be characterized based on its structural age rather than its chronological age. Based on the structural age and type of an individual's skin, cosmetic, dermatologic, medicinal or manipulative treatment may be customized. The method is based, in part, on quantification of discontinuities that arise in the skin during normal facial expression. The methods provide for evaluating changes in human skin response that occur over a short term (one day) or long term (one or more years). The methods provide for evaluating the skin's response to cosmetic, dermatologic or medicinal treatment or to any other factor alleged to affect the skin.

### FIELD OF THE INVENTION

The present invention pertains to the fields of cosmetics and dermatology, specifically to methods of quantifying structural changes in skin due to a variety of factors.

### BACKGROUND

Human skin is affected by exogenous or endogenous factors, many of which are deteriorative while some are presumed to be beneficial. These factors include gravity, topical dermatologic, sun exposure, pollution, smoking, second hand smoke, pharmaceuticals, oral supplements, diet, exercise, trauma, mechanical manipulation (i.e. massage) and chronological aging. Structural changes in the skin that are associated with some of these factors, include the deterioration of the collagen and elastin network in the surface layers of the skin. This deterioration causes loss of skin elasticity and firmness, leading to sagging of the skin. Also, as the skin ages, humans may develop permanent contraction of small facial muscles under the skin. In humans, the facial muscles are directly connected to the overlying skin through something called the superficial musculoaponeurotic system. The musculoaponeurotic system is what gives humans the ability to create a variety of facial expressions. However, once sufficient skin elasticity is lost, the permanent contraction of small muscles under the skin manifests as wrinkles in the skin, particularly between the eyebrows, near the outer corners of the eyes and at the corners of the mouth. The loss of collagen and elastin is further exacerbated by gravity, which pulls at the skin throughout the day. Elastically-compromised skin may be incapable or not fully capable of holding itself up against gravity. This commonly results in jowls and drooping eyelids.

Changes in mechanical properties of skin, from whatever cause, are not generally isotropic nor homogeneous in the affected area. A wrinkle then, is the result of localized changes in the mechanical properties of the skin and wrinkled skin may be thought of as being weaker than the surrounding skin. Skin wrinkles are qualitatively different from fine lines in the skin. Fine lines are the result of habitual facial expressions and occur in otherwise healthy skin. Fine lines are not related to permanent muscular contraction beneath the skin.

Methods for quantifying and characterizing the texture and appearance of human skin include macroscopic and microscopic techniques. Macroscopic techniques often involve a subjective assessment by a human agent. The downside to this is that there will always be a degree of uncertainty in assigning graded values to physical features based on human observation, no matter how well trained a human agent may be. To overcome this, various instrument-aided techniques have been developed that remove some or all of the human element, thus lessening the uncertainty. Techniques that rely on optical instrumentation are known and the simplest optical instrument is the camera. Photographs of test subjects are taken so that the image may be analyzed rather than analyzing the test subject directly. This has the benefit of capturing the physical features in a fixed form so they can be analyzed over an extended period of time. Measurements may be taken directly from the photograph; for example, the length of wrinkles in the skin may be accurately determined. Alternatively, the features under investigation may be identified on the photographic image and then classified within a previously defined classification scheme. The act of classification may be made by a human agent or by optical equipment, which may include optical scanning and processing software. These techniques say little or nothing about the mechanical properties of the skin itself and they are most useful only if a statistically meaningful scale has been previously defined. (See, for example, “Comparison of Age-Related Changes In Wrinkling and Sagging of the Skin In Caucasian Females and In Japanese Females”; Tsukahara et al.; Journal of Cosmetic Science; July/August 2004, vol. 55, no. 3, pp. 373-385.)

In contrast, mechanical properties of skin and other soft tissues have been investigated using various techniques common in mechanical engineering and materials testing. Many of these techniques measure the surface displacement and strain of a test sample under constant load. From these measurements, intrinsic properties such as elasticity, Young's modulus, tensile strength and hardness may be derived. These techniques have even been applied to living tissue with the aim of finding local discontinuities in the tissue. Such discontinuities may be indicative of a pathological process at work, altering the mechanical properties of the tissue. Some measurements of this type use invasive contact methods and equipment generally associated with materials testing, for example, a durometer for hardness testing, a strain gauge for tensile testing, suction cup and torsional methods for elasticity, etc. Often, it is not practical to perform these tests in vivo.

Less invasive methods of measuring mechanical properties of skin include digital image correlation. Various forms of digital image correlation have been developed, but generally, they all seek to measure the displacement and deformation gradients caused by a load applied to a surface. They do this by correlating small regions of a digital image made after deformation with those same regions on a digital image made before deformation. When this correlation is carried out at many points over the whole image of the specimen under investigation, it yields a vector displacement field for the deformed surface. From this displacement field, stress, strain and Young's modulus may be computed.

One digital image correlation technique, in particular, is digital image speckle correlation. Digital image speckle correlation (DISC) has been in use and development for more than two decades to analyze the response of materials to stress and the environment. In principle, all types of materials, living and non-living, may be studied with DISC. Generally, geometric features are identified in the field of a digital image before deformation and then these features are tracked to their new location in the image field after deformation. By this tracking, a vector displacement field for the deformed surface can be constructed. In the conventional method of DISC, reflective materials (speckles) are randomly distributed on the surface under examination. The speckles provide easy-to-track geometric features on the surface of the test specimen. After capturing one digital image of the undeformed surface and one digital image of the deformed surface, the images are divided into subsets. The subsets on the image of the undeformed surface are matched to the corresponding subsets on the image of the deformed surface. This is done through sophisticated numerical computer analysis, comparing patterns of light intensity in the before and after photos. The coordinates of the center points of each pair of subsets define a displacement vector which describes the average displacement of the subset as a result of the deformation. The displacement vectors can be resolved into vertical and horizontal components and that information may be represented as vertical and horizontal projection maps. Using numerical differentiation, the normal strain along either direction may be obtained.

In “Determining Mechanical Properties of Rat Skin With Digital Image Speckle Correlation” (Guan, et al., Dermatology, vol 208, no. 2, 2004, p. 112-119), the contents of which are herein incorporated by reference, there is described an in vitro application of DISC on samples of rat skin. Three sections of skin were tested, freshly excised skin, skin allowed to rest 24 hours after being excised and skin pre-treated for 24 hours with a commercially available cosmetic anti-wrinkle moisturizer. The skin sections were stretched in a tensile testing machine at a constant rate of 0.508 mm per minute. The speckle material consisted of 24 μm silicon carbide and talc material, which provide a high contrast black and white surface. Digital images were taken with a Kodak MegaPlus 1.6i charged-coupled device camera, having a resolution 2,029×2,048 pixels. For each skin sample, the tensile stress, tensile strain, ultimate strain, Young's modulus and break strength were determined. The article concludes, in part, that the moisturizer efficiently slowed down the loss of elasticity in the rat skin. The article further suggests, but does not describe, the use of DISC, in vivo, to monitor changes in skin elasticity, which may provide a means of predicting wrinkle formation. The article merely mentions, but does not describe, that the skin may be put under stress using a gas loading electrodynamometer. The article also suggests, but does not elaborate, that cosmetic efficacy may be measured by an in vivo DISC technique, where DISC measurements are made before and after the skin is treated with an anti-aging product. This reference does not disclose or suggest the in vivo techniques of the present invention, nor the method of the present invention for evaluating the effects of exogenous and endogenous factors on the skin.

A modified DISC technique has been successfully applied in vivo, using the pores of the skin for tracking deformation, rather than speckle material. (See, “Dynamic Facial Recognition With DISC: Identify the Enemies”, paper presented at the meeting of the American Physical Society, Mar. 22-26, 2004, Montreal). The musculature under the skin of the face provided the deformation of the skin and this reference describes a successful facial recognition method. The reference mentions that the technique may be used for early detection of skin disorders or skin abnormalities, but no further disclosure is made. This reference does not disclose or suggest the in vivo techniques of the present invention, nor the method of the present invention for evaluating the effects of exogenous and endogenous factors on the skin.

In “Investigations of Facial Recognition and Mechanical Properties of Aging Skin Through Digital Image Speckle Correlation” (submitted to the Intel Science Talent Search, November, 2004) there is disclosed an in vivo application of DISC technology to human facial skin. It was determined that age-related changes in the skin (for example, loss of elasticity) can be observed by Examining a cross section of a vector displacement map. The map is created from vector displacement data obtained in a DISC-like procedure.

None of the foregoing discloses the use of a non-invasive, in vivo, DISC-type data collection system, to quantify, qualify or otherwise evaluate the effects of one or more exogenous or endogenous factors on the skin.

### OBJECTS OF THE INVENTION

A main object of the present invention is to provide a non-invasive, in vivo method of characterizing the behavior of human skin during normal facial expression.

Another object is to evaluate the structural age of human skin based on discontinuities in the skin that arise during normal facial expression.

Another object is to provide a method that evaluates the effects of exogenous or endogenous factors on the skin.

Another object is to provide a method for evaluating changes in human skin response that occur over the short term (one day) or long term (one or more years).

Another object is to provide a method for characterizing the human skin response to cosmetic, dermatologic, medicinal or mechanical treatment and thereby evaluating the efficacy of such treatment.

Another object is to provide a method of prescribing a skin treatment regimen.

### DESCRIPTION OF THE FIGURES

FIG. 1 is a schematic representation of a digital image speckle correlation system used in the present invention.

FIG. 2 is an example a vertical projection map.

FIG. 3 is a cross section o taken along line A-A of FIG. 2.

FIG. 4 is the a graph of cross sections taken along a line of symmetry between the eyes, through the vertical projection maps of the three test subjects in Example 2.

FIGS. 5a and 5b respectively, are vector displacement maps made from one “younger” and one “older” test subject, the area of study being the region immediately lateral to the outer canthus if the eye.

FIGS. 6a and 6b are cross section graphs corresponding to FIGS. 5a and 5b, from which the full width at half maximum may be measured and used as an indicator of skin structural age.

### SUMMARY OF THE INVENTION

The present invention uses a non-invasive, in vivo form of digital image speckle correlation to track deformation of human skin during normal muscular contraction. The skin and musculature of the face are of particular interest, but the present invention may be applied to any part of the body and to non-humans. Unlike in vitro methods and unlike invasive, in vivo methods that tension the skin with an apparatus, the present invention relies on normal muscular function to create before and after deformation images. From those images, it is possible to develop quantitative and qualitative characterizations of skin. For example, skin may be characterized based on its structural age, rather than its chronological age. It is also possible to characterize how the skin of an individual changes over long and short term and how the skin responds to cosmetic, dermatologic or medicinal treatment or to any other factor alleged to affect the skin. Meaningful comparisons of the skin of different persons is possible, including comparisons of persons with the same or different skin types. Based on the structural age and type of an individual's skin, cosmetic, dermatologic, medicinal or manipulative treatment may be customized.

### DETAILED DESCRIPTION OF THE INVENTION

The present invention exploits the close connection that exists between the facial muscles and the overlying skin through the superficial musculoaponeurotic system. It does this by using the facial muscles to deform the skin, rather than using an externally applied load. There are several benefits to doing this. Firstly, the DISC technique of the present invention is much simpler than one that requires applying an external tensile load to the skin, particularly in the areas of study, where wrinkles commonly form. Generally, it may not even be practical to apply such loads. In contrast, the DISC technique of the present invention is in vivo, while being completely non invasive. Furthermore, an externally applied load would tension the skin in an unnatural manner. The response of the skin to an external load may bear little or no resemblance to the natural response that the facial skin undergoes due to muscular activity. So while a DISC technique that uses an externally applied load may be useful for measuring some physical parameters of the skin, like, Young's modulus, such a technique misses the opportunity to characterize the dynamic behavior of the skin itself, in a real life situation. Facial muscular movements and the resulting response of the skin are highly characteristic of individuals. Therefore, measuring a mechanical property like Young's modulus or some other material parameter, does not give one the ability to predict the behavior of the skin nor the skin's response to treatment, because the system is far too complicated and specific to each individual. In contrast, the technique of the present invention directly measures the dynamic response of an individual's skin during normal movements. Therefore, the techniques of the present invention not only avoid the complexity of relating the skin's mechanical properties to its dynamic response, but the techniques of the present invention incorporate the dynamic response of an individual's skin into quantitative and qualitative characterizations of skin. This is a great advantage because the skin's dynamic response is part of what creates the individual's appearance to the rest of the world. Because people almost never hold their faces motionless during the day, it makes sense to utilize the characteristic movements of each individual when evaluating or characterizing that person's skin.

Throughout the specification, the terms “structural age” and “structurally older” concern the condition of the skin and degree of deterioration of the skin, and are used to distinguish over “chronological age,” which refers to the length of a person's life, regardless of the condition of his or her skin. Some older persons may have structurally young skin and vice versa. The present invention is concerned, in part, with assigning a structural age to a person's skin regardless of his or her chronological age.

Throughout this specification, the terms “comprise,” “comprises,” “comprising” and the like, shall consistently mean that a collection of objects is not limited to those objects specifically recited. Also, the term “normal facial expression” refers to the motion of the skin caused by facial muscles, as opposed to motion caused by an externally applied load.

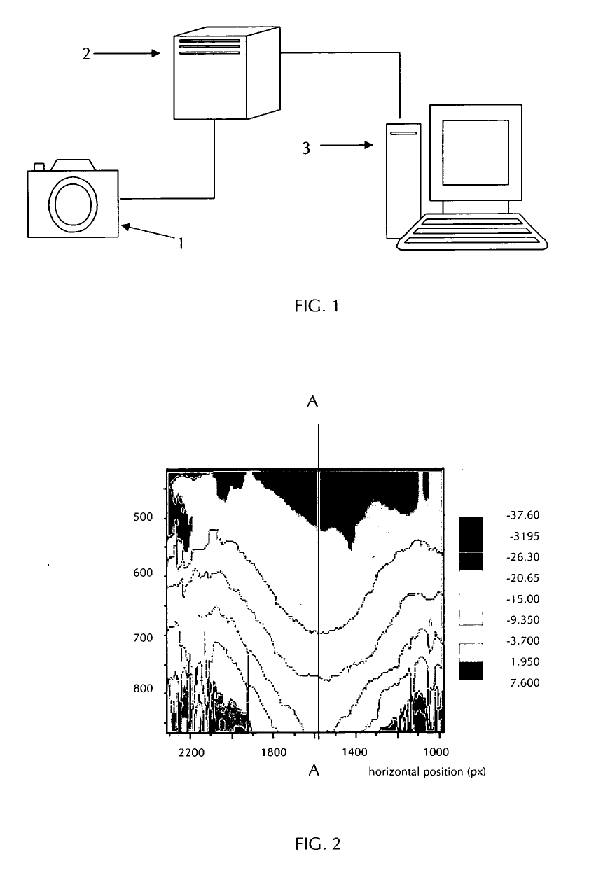

FIG. 1 is a schematic representation of a digital image speckle correlation system used in the present invention. A camera (1) for capturing digital images is a charge-coupled device providing a minimum of four mega pixel resolution. This resolution is sufficient to resolve the pores of human skin, which are the points being tracked in the technique of the present invention. Technically, virtually any feature in the image field may be useful for tracking between images, however, the success of a DISC-type technique depends on having a plethora of features to track. In humans, skin pores fulfill this requirement. Some useful cameras are Canon EOS Rebel Digital camera (6.3 mega pixel resolution) and the Toshiba DK-120F CCD camera. Data collected by the camera is pre-processed by a frame grabber (2), such as PIXCI® from EPIX®, and the digitized information is downloaded to a computer (3) for numerical analysis. Many research groups have developed their own software on the DISC technique to suit their own needs. Persons of ordinary skill in the art are capable of developing such software without undue burden. Furthermore, there are also commercially available software applications, one being VIC-2D from Correlated Solutions Inc., (West Colombia, S.C.), with an advertised displacement accuracy of better than one one-hundredth of a pixel. Another supplier of digital image correlation systems and software is Optical Metrology Innovations, Cork, Ireland.

A typical procedure comprises capturing two images. The second image is made shortly after the first, after the skin of the test subject has been deformed. For some studies, this procedure may be repeated at a later time. Throughout the specification, “deform” means that the skin has assumed a shape that is different from an initial shape. In the present invention, deformation is accomplished by normal muscular contraction of those muscles below the general area of skin under examination. For example, if the area of study is at the corner of the mouth, the “before” or “initial” or “undeformed” image may be of a neutral facial expression, with minimal muscular tensioning of the skin. The “after” or “final” or “deformed” image may be the subject smiling or holding an object in his/her teeth. Or perhaps, the area under study is the forehead, for which an initial image may be a neutral facial expression with the eyes closed, while a final image may be with the eyes opened and raised. In general, the deformed image is made by engaging those facial muscles that are under the area of skin being studied, so that the skin in question undergoes some deformation. Preferably, the head of the subject is held motionless during image capture. For example, a chin rest or a full head harness may be used to hold the subject's head still. It is also preferable that the camera be held immovable during picture taking. A camera stand may be used for this purpose.

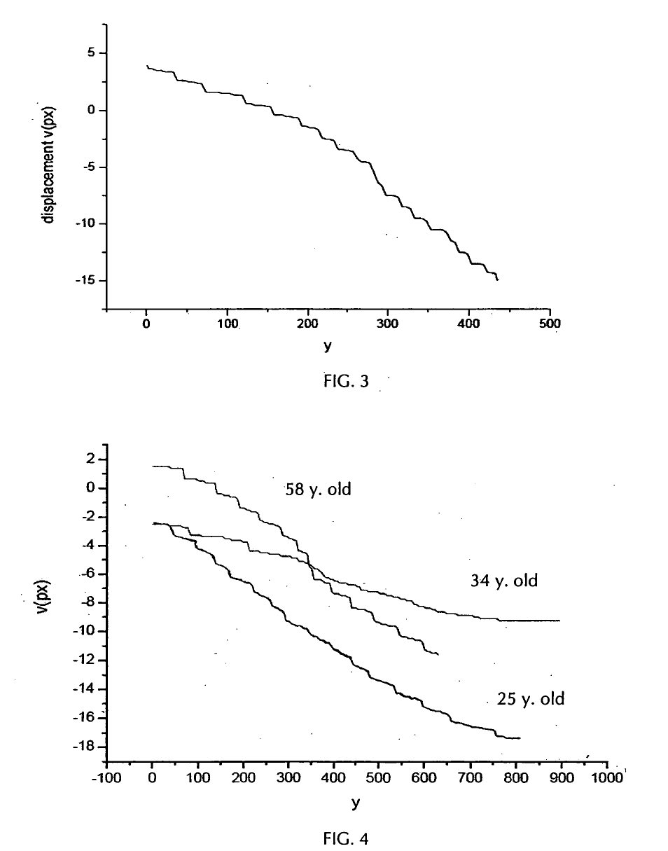

Once the images are acquired, software, such as Photoshop© from Adobe®, is useful for imposing on the images, a boundary of the area to be studied and a reference coordinate system, as well as for obtaining a rough estimate of pore displacement. The boundaries are somewhat arbitrary and may be chosen to define a domain large enough for analyzing several areas of the skin. The imaging software determines the coordinates of each pore in the displacement field relative to the reference coordinate system, for the before and after image. From this data, correlations are established between the pores in the before and after images and a field of displacement vectors, as discussed above, is generated. Each displacement vector represents the movement of one pore from its initial to final location. Each pore vector in the field of displacement vectors is resolved into its vertical and horizontal projections, from which vertical and/or horizontal projection maps are produced. An example of a field of displacement vectors generated by the DISC technique of the present invention is shown in FIG. 2. FIGS. 3a and 3b respectively show the vertical and horizontal projection maps of the vector field of FIG. 2. In the projection maps, the horizontal and vertical axis convey the coordinates of any position in the field of study. In FIGS. 3a and 3b, the units are pixels. Areas of constant displacement are color coded in these figures. From one or more of the projection maps, a cross section is taken through an “interesting” area of the map. An “interesting” cross section is one that passes through an area of relatively large displacement or steep gradient as viewed on the projection maps. Surprisingly, the use of cross section graphs made from one or more projection maps has proven very useful in characterizing human skin in a variety of situations. The following non-limiting examples may increase the reader's appreciation of the present invention.

### EXAMPLE 1

Pore Displacement, Structural Age of Skin and Wrinkle Prediction

In this example, the area of study was the forehead. An initial image was made with the eyes closed and the final image was made with the eyes opened and looking up. Both images were made with minimal contraction of the muscles of the forehead. A field of displacement vectors was generated and the vertical component of that field, or projection map, is shown in FIG. 2, where each shade represents the indicated displacement in pixels. For this deformation, the displacement of the pores of the forehead is predominantly vertical and there is a vertical line of approximate symmetry between the eyes. Because of this, it is convenient to look at a cross section of the vertical projection map, the cross section being made along the vertical line of symmetry between the eyes. A graph of vertical displacement along this cross section is shown in FIG. 3, where the vertical displacement of the pores, in pixels, is shown on the vertical axis and the vertical component of the initial position of the pores, in pixels, is shown on the horizontal axis.

From the shape of the graph of FIG. 3, it may at once be appreciated that the vertical displacement of skin occurs in steps. Further examination of the images reveals that the vertical portions of the steps occur at fine lines and wrinkles in the skin. In performing the described movement, those pores located between wrinkles displace comparatively little, while those near a wrinkle displace significantly more. Apparently, the tension supplied by the underlying muscle propagates through the skin in an anisotropic manner, causing greater strain nearer to wrinkles and lesser strain further away from wrinkles. Furthermore, it has been observed that the larger the vertical step (pore displacement) in FIG. 3, the deeper the wrinkle. This may make sense when it is remembered that wrinkles are localized areas of weakened skin surrounded by stronger skin. The same weakness that accounts for the depth of the wrinkle may also account for, or at least be correlated to, the excessive displacement of the skin at the wrinkle. Therefore, the presence of localized discontinuities in pore displacement (which show up as steps on a cross section of a vector projection map) during normal facial expression is herein, identified as a sign of ageing skin. Surprisingly, we are led to the suggestion that step-wise displacement of skin pores, as a result of normal facial expression, may be used to characterize the structural age of the skin. The greater the extent of localized discontinuities, the older is the skin from a structural point of view. Therefore, this example establishes an in-vivo method of characterizing the skin of a human individual comprising the step of identifying discontinuities in pore displacement during normal facial expression.

Furthermore, and completely unexpected, identifying localized discontinuities in pore displacement during normal facial expression is useful even before well developed wrinkles are visible. This is because both weakened skin and permanent contraction of the small facial muscles are involved in wrinkle formation. The skin is weakened by exposure to harsh exogenous and endogenous factors, while the condition of muscular tetany arises from other causes. Structural ageing or weakening of the skin is generally ongoing and significant effects will generally accrue well before the permanent contraction of the small facial muscles. Therefore, localized discontinuities in pore displacement develop and are observable well before wrinkles develop. Therefore, while the techniques of the present invention are useful for characterizing skin of any condition, it is important to emphasize that discontinuities in pore displacement may be observable even when there are absolutely no wrinkles in the skin. It's not that the wrinkles are too small to be noticed, it's that they may not be present at all. Therefore, by measuring localized discontinuities in pore displacement during normal facial expression, the future location of wrinkle formation may be predicted. This is unknown in the prior art.

This principle is new and may extend beyond the DISC technique described herein. For example, any quantitative and/or qualitative determination of discontinuity in pore displacement during normal facial expression will be useful for characterizing the structural age of the skin and for predicting wrinkle formation. However, the pore-DISC technique used herein is particularly convenient. In the forehead example, above, the pore displacement is predominantly in one direction (vertical) and identifying discontinuities in pore displacement was relatively simple. Other areas of the face present more complex patterns of pore displacement, which generally depends on the shape and action of those muscles that are recruited to perform the movement. Nevertheless, if the structure of the skin has developed anisotropic weaknesses as a result of exogenous and endogenous factors, those weaknesses will show up as discontinuities in pore displacement.

### EXAMPLE 2

An Age-Correlation Study—Forehead

The following experiment was carried out on three persons, aged 25, 34 and 58. These test subjects “looked their age”. Images were made of the forehead area, as described above. The vector displacement maps were resolved into horizontal and vertical projection maps and the vertical projection map was analyzed by studying a cross section along the line of symmetry between the eyes. FIG. 4 shows the vertical cross section graphs for all three subjects. The units on both the horizontal and vertical axes are pixels. As above, the most critical observation is the presence of steps in the graphs, these steps corresponding to localized areas of skin weakness. Clearly, localized areas of skin weakness are present even in a twenty-five year old with apparently firm, young skin. Therefore, the presence of local discontinuities (steps) in pore displacement due to normal facial expression does not by itself allow a comparison of one test subject to another. Another means of comparing one subject to another is needed. To that end, in FIG. 4, the step sizes were measured to determine the maximum and minimum step size, from which was obtained their ratio. This info is shown in table 1.

TABLE 1
Step size in cross section of vertical projection map used to infer skin
weakness (ageing)

| Test subject age | Maximum step size | Max/Min Ratio |
|---|---|---|
| 25 | 0.52 | 0.3 |
| 34 | 0.62 | 0.5 |
| 58 | 1.39 | 1 |

The data suggests that structural aging is accompanied not only by increased step size, but also by increased variation in step sizes. The interpretation of this is that structural aging may be accompanied by not only more localized areas of skin weakness, but by increased variation in the weakness of those areas. Structurally younger skin, in contrast, has much lesser variation in localized weaknesses. This means that in a structurally older person with well developed wrinkle formation, there are not only more wrinkles, but more variation in the depth of those wrinkles. Prior to wrinkle formation, as the skin ages, it develops more weak areas and, perhaps expectedly, more variation in the weakness of those areas. It may be that those areas of weakness that developed earlier in life are the weakest areas of the skin, while more newly formed areas of weakness have not weakened as much. Therefore, it is surprisingly found that structural age may be distinguished not only by step size on the vector displacement graphs, but also by the variation in the step sizes.

This knowledge may be useful in a number of ways. For example, two persons of similar chronological age and similar skin appearance may be analyzed for variation in displacement of pores during normal facial expression. From such an analysis, it may be determined that one subject has a particular ratio of maximum to minimum pore displacement while the other has a ratio that is three times that of the first. Although, these subjects are similar in appearance and chronological age, these measurements would predict that the pace of wrinkle formation once it begins will be faster for the second subject, the one with greater variation in pore displacement. With that knowledge, the second subject could take preventive action to slow the structural weakening of the skin. The efficacy of such treatment could be evaluated by comparing the pore displacement data before and after treatment. So, not only may the skin of different individuals be compared, but the skin of an individual may be compared to itself at different times. If the variation in pore displacement (and therefore, skin weakness) continues to grow, then treatment is not effective. If the variation remains steady or decreases, then treatment may be having an effect.

### EXAMPLE 3

A Study on the Effects of Gravity—Forehead

As a further example of its usefulness, the technique of the present invention was used to study the effects of gravity on the facial skin, particularly the skin of the forehead. Generally, the skin and muscles of the face are subject to the pull of their own weight. This weight may be at a maximum for sixteen or more hours per day while the head is in an upright position.

In the first variation of this study, two sets of before and after images were made. The subject was in a laying down, face up (supine) position. By laying down, the effects of gravity are somewhat neutralized because the skin of the forehead does not have to support its own weight. After this, two more images were taken, the only difference being that the subject was in an upright position, standing, for example. In each case, the before and after images were made within several seconds of each other. The before expression was with the eyes closed, but otherwise no muscular involvement. The after expression was with the eyes opened and looking up. A chin rest was used to stabilize the head. As above, cross sections of the vertical displacement maps were measured to determine a minimum and maximum step size and their ratio. This procedure was performed on one “young” and one “old” test subject. The skin of the younger test subject appeared firm and without wrinkles. The skin of the older test subject was clearly structurally older, with well developed wrinkle formations. The data is shown in table 2. As the data show, the effect of gravity is relatively small for the structurally younger skin. For the older person, the immediate effect of gravity was to increase by 240%, the variation in pore displacement. We note that the percent change for the younger test subject is negative. This may be due to the test uncertainty being larger than the measured effect and the result indicates that the immediate effect of gravity for “younger” skin may not be not significant.

TABLE 2
Two gravity studies

| | Max/Min Ratio | | |
|---|---|---|---|
| Test subject | Laying Down | Standing Up | % Change |
| young | 5 | 4 | −20 |
| old | 0.4 | 10 | +240 |

| | Standing Up a.m. | Standing Up p.m. | |
|---|---|---|---|
| young | 1 | 1 | 0 |
| old | 5.5 | 10 | +82 |

In a second variation of this study, a first set of before and after images were made in the morning (about 9 a.m.), with the subject in the standing up position. A second set of before and after images was made in the afternoon (about 4 p.m.), about seven hours after the first, also with the subject in the standing up position. This was done for the same “young” and “old” test subjects above. Prior to the morning measurements, the effects of gravity were mitigated for several hours because the test subjects were reposed for sleep throughout the previous evening. Therefore, at the start of this test, the skin was well rested.

In this case, the younger subject had a max/min pore displacement ratio of 1, in both the morning and afternoon tests (see table 2). In contrast, the older subject had a max/min ration of 5.5 in the morning and 10 in the afternoon, when gravity had been at work for an extended period of time. That's an 82% change that accumulated over several hours. These results certainly suggest that the effects of gravity over the course of one day are much more pronounced in structurally older skin than structurally younger skin. Over several hours, gravity had little or no effect on the response of the structurally younger skin. In contrast, the structurally older skin was significantly weaker after several hours of exposure to gravity. This demonstrates that gravity, even over a short term (one day or less), can have a significant effect on the skin's response to normal facial expressions.

While the weight of the skin may seem inconsequential, this example shows that gravity has both immediate and accumulated effects in the skin of structurally older persons. Therefore, we can use the foregoing study to distinguish between test subjects who appear similar in structural age by using gravity to magnify changes in the max/min ratio of pore displacements. For the test subject with structurally older skin, we expect to see a more dramatic change in that ratio than for the subject with the structurally younger skin. Once the structural age of the skin has been assessed, a treatment more appropriate to that age may be undertaken. Thus, persons whose skins appear similar, may actually be in need of different treatment. This test protocol is advantageous because it offers another view of the skin's response to normal facial expression, while remaining completely non-invasive.

Interestingly, this study also suggests that people who do not get the medically recommended amount of sleep are harming their skin as a result of being in an upright position for more hours of the day than they otherwise would be. This harmful effect is apart from any effect caused by insufficient sleep, which, by the way, could also be studied by the methods of the present invention. Finally, these studies suggest that valuable information about the effects of gravity on the structural age of skin may be gained by performing the measurements before, during and after an extended stay in a reduced gravity environment, as in Earth orbit or on the moon, for example. Thus a novel, in-vivo method of quantifying the effects of gravity on the skin could comprise the following steps: using a DISC-type system to generate an initial displacement map from a patch of skin subjected to a first net gravitational force; using a DISC-type system to generate a final displacement map from the patch of skin subjected to a second net gravitational force; generating cross section displacement graphs from the initial displacement map and from the final displacement map; identifying the maximum and minimum displacements on each cross section graph; computing initial and final cross section displacement ratios for the corresponding cross section displacement graphs; comparing the initial and final cross section displacement ratios.

### EXAMPLE 4

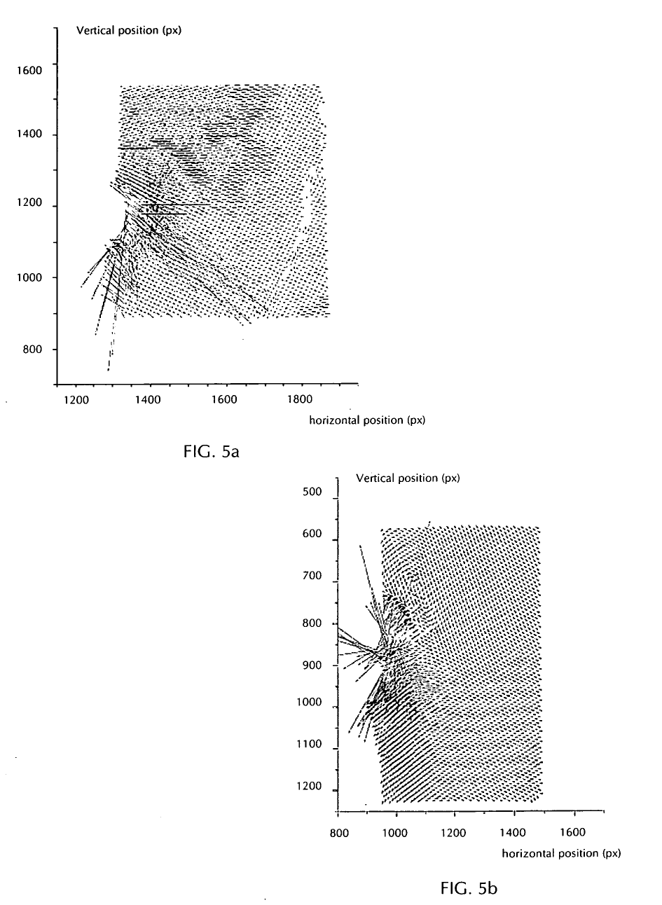

Stress Propagation Skin Analysis—Eye Area Ten panelists were tested according to methods described herein, over and area of 532 pixels×652 pixels, immediately lateral to the outer canthus if the eye. The first picture was taken with the eyes opened and relaxed, while the second was taken with the eyes closed with minimal pressure. This motion is effected by the orbicularis oculi muscle, predominantly. In the area of study, the fibers of the orbicularis oculi muscle are aligned generally vertically. FIG. 5 shows two vector displacement maps, 5a of a “younger” test subject and 5bB of an “older” test subject. In these maps, each vector corresponds to the movement of one point of the surface of the skin. It can be seen that, in the area of study, away from the region of highly concentrated stress, the skin of the younger person displaces basically horizontally (parallel to the X axis). In comparison, the displacement of the skin of the older person significant horizontal and vertical (parallel to the Y axis) components. This may be interpreted by saying that the skin of the older person is less flexible in the horizontal direction than that of the younger person. As a result, the stress induced by the musculature is more spatially concentrated and falls off faster in the older person than it does in the younger person, while also pulling the skin of the older person in many directions. In the younger person, the stress induced in the more flexible skin is able to spread out horizontally and reduce more gradually than in the older person.

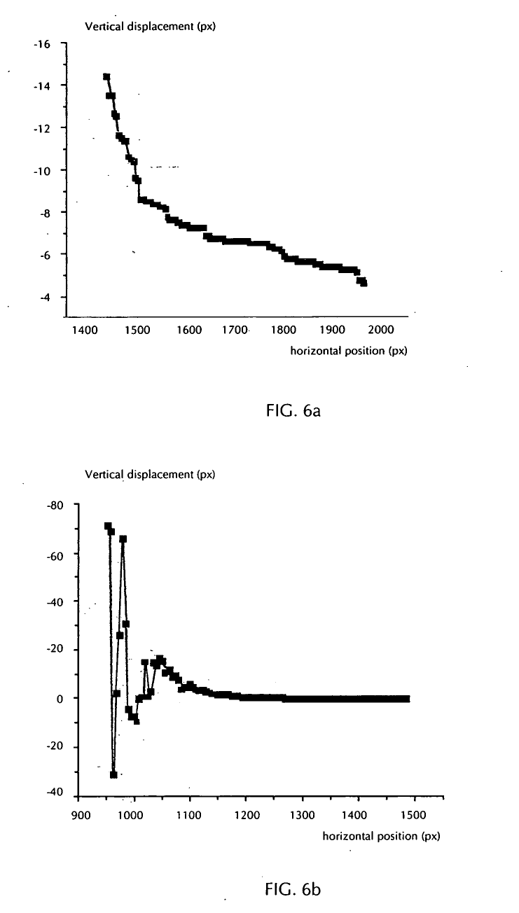

For each test subject, a horizontal displacement map was created and a cross section through the horizontal displacement map was plotted. The cross section plots for the “old” and “young” subject of FIGS. 5a and 5b are shown in FIGS. 6a and 6b. To provide some comparative measure of the rates at which the stress propagates through the skin, the full width at half maximum (FWHM) was measured from each cross section graph. A larger FWHM indicates that the stress is spreading out and falling of more slowly, i.e. more flexible, younger skin. The results are shown in table 3, which divides the ten panelists in to two groups: five younger panelists, ages 18-23, and the five older panelists, over 55 years of age. As can be seen, there is a dramatic difference in the behaviors of older and younger skin, evidenced by the significantly larger FWHM of the younger panelists. Thus, the FWHM is identified as a parameter that may be correlated to structural age of skin. More generally, the method of generating vector displacement maps in vivo, for the purpose of correlating stress propagation parameters to a structural age of skin is called, herein, stress propagation skin analysis.

TABLE 3
full width at half maximum

| FWHM - young | FWHM - old |
|---|---|
| 63 | 27 |
| 53 | 8 |
| 60 | 7 |
| 163 | 7 |
| 112 | 15 |
| Average = 90 | Average = 13 |

### EXAMPLE 5

Stress Propagation Analysis—Cheek Area

In this example the area of study was a rectangular section of the cheek, 1000 pixels×2000 pixels, immediately lateral to a corner of the mouth. Thirteen panelists ranging in age from 20 to 59 years were tested. The deformation is a natural deformation of the skin of the cheek, caused by opening the mouth. A first image was acquired with the mouth closed and relaxed. A second image was acquired with the mouth slightly opened with minimal effort. For better control during acquisition of the second image, each subject held a tongue depressor between her teeth. For each panelist, one set of images was acquired in the morning and another set about twenty-four hours later. As above, the full width at half maximum was measured from a cross section graph of the vertical displacement map. The results for both days were averaged and are shown in table 4.

TABLE 4
FWHM Stress Propagation Analysis

| Age | FWHM |
|---|---|
| 20 | 210 |
| 23 | 255 |
| 24 | 205 |
| 30 | 270 |
| 34 | 220 |
| 37 | 205 |
| 40 | 145 |
| 43 | 175 |
| 44 | 160 |
| 46 | 185 |
| 47 | 105 |
| 57 | 180 |
| 59 | 180 |

Consistent with example 4, the data in table 4 show that FWHM generally decreases with increasing age. It also shows that a comparatively steep decrease in FWHM occurred between about 35 and 50 years of age. This steeper region of the chart suggests that in the population at large, skin may not age at a steady rate.

### EXAMPLE 6

Produce Efficacy Study—Cheek Area

On this example the area of study was a rectangular section of the cheek, 400 pixels×1000 pixels (1 pixel corresponding to about 60 microns), immediately lateral to a corner of the mouth. Nineteen panelists ranging in age from 20 to 63 years were tested. The deformation is a natural deformation of the skin of the cheek, caused by opening the mouth. A first image was acquired with the mouth closed and relaxed. A second image was acquired with the mouth slightly opened with minimal effort. For better control during acquisition of the second image, each subject held a tongue depressor between her teeth. For each panelist, one set of images was acquired in the afternoon of day 0 and another set was acquired in the afternoon of day 30. Following the initial measurements on day 0, each panelist was to apply a once-per day topical skin treatment product, until the second set of images was acquired at day 30. Ten panelists completed the study. As above, the full width at half maximum was measured from a cross section graph of the vertical displacement map. The raw data for both days are shown in table 5. Panelist aged 47 is a statistical outlier at day 30. After eliminating the results of that panelist, the average percent change in FWHM after 30 day treatment was 35%, ranging from 2% to 149%.

TABLE 5
Product efficacy study, FWHM raw data

| Age | FWHM (day 0) | FWHM (day 30) |
|---|---|---|
| 20 | 192 | 233 |
| 30 | 130 | 182 |
| 37 | 185 | 256 |
| 40 | 152 | 159 |
| 43 | 179 | 182 |
| 44 | 108 | 159 |
| 46 | 67 | 167 |
| 47 | 54 | 730 |
| 58 | 169 | 189 |
| 59 | 154 | 161 |

TABLE 6
Product efficacy study, FWHM percent change

| Age | FWHM % change |
|---|---|
| 20 | 21% |
| 30 | 40% |
| 37 | 38% |
| 40 | 5% |
| 43 | 2% |
| 44 | 47% |
| 46 | 149% |
| 58 | 12% |
| 59 | 5% |
| average | 35% |

These results further establish the DISC method described herein as a novel tool for quantifying and qualifying the effects on the skin, over time, of virtually any exogenous or endogenous factor, as for example, a skin treatment regimen. The results in table 6 further demonstrate the need for such a tool because, among the panelists, the range of response to treatment was quite varied. This highlights the value of the methods described herein as a tool to customize treatment for specific individuals. For a given individual, the efficacy of a treatment may be evaluated based on data derived from vector displacement maps, as disclosed herein. With such information, an informed decision can be made about whether to continue the same treatment or to change the treatment protocol.

At this point, it will be appreciated that the techniques of the present invention directly measure the dynamic response of an individual's skin during normal movements and that information may be incorporated into other measures of skin reaction, such as the skin's reaction to exogenous and endogenous factors and such as the comparison of one skin to another for the purpose of determining the structural age of skin. This is a great advantage because the skin's dynamic response is part of what creates the individual's appearance to the rest of the world. Another great advantage of the present invention, is that the techniques are in vivo while being non-invasive.

The foregoing is not limited by the examples described herein and the techniques may be used to evaluate the effect on the skin and/or the skin's response to virtually any exogenous or endogenous factor. In fact, within the scope of the present invention, one could, by routine application of the principles described, accumulate enough information from one or more populations, such that the structural age of the skin can be meaningfully correlated to chronological age and other factors. One could imagine conducting test subject interviews and sampling statistically relevant sub-populations defined according to any factor of interest, including, chronological age, ethnicity, geographic region, lifestyle, gender, personal income, diet, exercise, etc. Data like that of table 4 could be generated for each sub-population and statistically meaningful correlations could be identified between the structural age of skin and various exogenous and endogenous factors. The correlated information for each sub-population could be presented in the form of charts, graphs or any convenient presentation format. This correlated information would have several uses. For example, valid conclusions could be drawn as to the relative harm or help to the skin caused by those factors. As a purely hypothetical example, for illustrative purposes only, one can imagine easily accumulating statistically significant data to support a statement like, “Three hours per week of sun exposure is ten times more harmful to the skin than smoking a pack per week of cigarettes, for persons between the ages of 25 and 40.” In another example of the potential use of such data, a person fitting the profile of a particular sub-population could place themselves, by age, on a chart or graph. The individual's position on the graph would then be an indicator of the future course of ageing of the individual's skin. For example, if the individual found that he/she was approaching the rapid skin ageing portion of the graph, the individual may be encouraged to take preventative action.

Another use of correlated data from various sub-populations as described, is the identification of causative factors of ageing and the ability to prioritize those factors during different stages of life. At different stages of life, the primary causes of skin ageing are likely to change. One might find expected results, like the skin of sun worshippers ages faster than that of persons receiving more moderate exposure. But given the number of potential factors, some hitherto unknown connections would undoubtedly come to light. Another use of the correlated data drawn from statistically relevant sub-populations is as a means of monitoring disease or treatment progression, especially diseases or treatments affecting the skin, like some system-wide accelerated ageing diseases. In this case, the structural age of the skin may be useful as a diagnostic tool to assess how a disease or treatment is progressing in some other system of the body.

Therefore, the completely non-invasive, in vivo techniques of the present invention may easily and usefully be extended to statistically significant populations and when done so, the relative importance of various exogenous and endogenous factors may be established by directly measuring their effects on the skin. This is unlike anything in the prior art.

That which is claimed is:

1. An in-vivo method of characterizing the skin of a human individual comprising the step of identifying in the skin, discontinuities that arise during normal facial expression.

2. The method of claim 1 wherein the discontinuities are discontinuities in pore displacement, the discontinuities being identified using a means for digitizing images and digital image correlation software.

3. The method of claim 2 wherein the means for digitizing images and digital image correlation software are part of a digital image speckle correlation system.

4. The method of claim 1 wherein the human skin is characterized in terms of a structural age and/or predicted wrinkle formation.

5. The method of claim 1 wherein the discontinuities are identified as a step pattern on a cross section graph of a vector displacement map.

6. A method of comparing the structural age of the skin of two or more individuals comprising the steps of:
carrying out the method of claim 5 for each individual,
measuring the ratio of maximum to minimum step size for each individual; and
identifying the structurally oldest skin as that with the greatest ratio.

7. A method of predicting the location of wrinkle formation comprising the steps of:
carrying out the method of claim 5 for an individual,
identifying the largest step sizes on the cross section graph of the vector displacement map; and
correlating the location of the largest step sizes on the vector displacement map to their locations on the skin.

8. A method of characterizing the skin of an individual in terms of its response to one or more exogenous and/or endogenous factors, the method comprising the steps of:
carrying out the method of claim 1 for the individual,
exposing the skin to one or more exogenous or endogenous factors for a period of time,
carrying out the method of claim 1 a second time; and
comparing the pore displacement data before and after the exposure.

9. The method of claim 8 wherein the one or more exogenous factors are selected from the group consisting of gravity, topical dermatologic, sun exposure, pollution, smoking, second hand smoke, oral pharmaceuticals, oral supplements, diet, exercise, trauma or physical manipulation.

10. The method of claim 9 wherein the topical dermatologic is an anti-aging agent, an antioxidant, an anti-inflammatory, an anti-cellulite, an anti-wrinkle agent, a sunscreen, a moisturizer, a moisture barrier or a collagen enhancer.

11. The method of claim 10 wherein the step of exposing the skin to a topical dermatologic includes multiple applications of the topical dermatologic to the skin over an extended period of time.

12. The method of claim 9 wherein the exercise is cardiovascular or myo-tonifying.

13. The method of claim 12 wherein the tonified muscles are facial muscles.

14. The method of claim 8 wherein the one or more endogenous factors include chronological aging.

15. The method of claim 8 wherein the one or more factors exert effects on the skin over a period lasting from about one second to several decades.

16. An in-vivo method of comparing the structural age of the skin of two or more individuals comprising the steps of:
having each individual repose in a laying down position for a period of time,
shortly after laying down, carrying out the method of claim 5 for each individual, with each individual in an upright position,
computing a first ratio of maximum to minimum step size for each individual;
having each individual repose in an upright position for at least about seven hours,
carrying out the method of claim 5 a second time for each individual, with each individual in an upright position,
computing a second ratio of maximum to minimum step size for each individual; and
identifying the structurally older skin as that with the greatest difference in first ratio to second ratio.

17. An in-vivo method of quantifying the effects of gravity on the skin comprising the following steps:
using a DISC-type system to generate an initial displacement map from a patch of skin subjected to a first net gravitational force;
using a DISC-type system to generate a final displacement map from the patch of skin subjected to a second net gravitational force;
generating cross section displacement graphs from the initial displacement map and from the final displacement map;
identifying the maximum and minimum displacements on each cross section graph;
computing initial and final cross section displacement ratios for the corresponding cross section displacement graphs;
comparing the initial and final cross section displacement ratios.

18. The method of claim 16 wherein the first or second net gravitational force is the result of the patch of skin being in a reduced gravity environment.

19. The method of claim 1 wherein the discontinuities show up as one or more concentrations of induced stress in the skin, the concentrations being identified using a means for digitizing images and digital image correlation software.

20. The method of claim 19 wherein the means for digitizing images and digital image correlation software are part of a digital image speckle correlation system.

21. The method of claim 19 wherein the human skin is characterized in terms of a full width at half maximum as determined on cross section graph of a vector displacement map.

22. The method of claim 21 wherein the areas of concentrated stress are identified as those regions having relatively small full widths at half maximum.

23. An in-vivo method of comparing the structural age of the skin of two or more individuals comprising the steps of:
carrying out the method of claim 22 for each individual,
identifying the structurally older skin as that with the smaller full width at half maximum.

24. A method of evaluating the efficacy of a skin treatment regimen, the method comprising the steps of:
carrying out the method of claim 22 on a portion of skin,
measuring a first full width at half maximum,
applying a skin treatment regimen to the portion of skin,
carrying out the method of claim 22 a second time on the portion of skin,
measuring a second full width at half maximum; and
comparing the full width at half maximum data before and after treatment.

25. An in-vivo method of characterizing the skin of a human sub-population comprising the steps of:
identifying a human sub-population by one or more traits common to members of the sub-population,
from a statistically meaningful number of sub-population individuals acquire personal information about the individuals,
identifying in each individual's skin, discontinuities that arise during normal facial expression,
correlating the discontinuity information to the individual's personal information.

26. A non-invasive, in vivo method of determining the relative importance of the effects of various exogenous and endogenous factors on the skin of an individual, the method comprising the steps of:
providing one or more presentations of sub-population data on the discontinuities that arise during normal facial expression, each presentation of being correlated to one or more exogenous and endogenous factors,
identifying one or more presentations of data appropriate to the individual,
locating by chronological age, the individual's position on each presentation, and
determining the relative importance to skin ageing of each exogenous and endogenous factor.

27. A method of correlating stress propagation parameters to a structural age of skin, the method using stress propagation skin analysis.
